(19) United States
(12) Patent Application Publication
(10) Pub. No.: US 2024/0049969 A1
(43) Pub. Date: Feb. 15, 2024

(54) ELECTRONIC DEVICE AND OPERATION METHOD OF THE SAME

(71) Applicant: SAMSUNG DISPLAY CO., LTD., YONGIN-SI (KR)

(72) Inventor: Chul KIM, Yongin-si (KR)

(21) Appl. No.: 18/295,506

(22) Filed: Apr. 4, 2023

(30) Foreign Application Priority Data
Aug. 10, 2022 (KR) 10-2022-0099642

(51) Int. Cl. A61B 5/021; A61B 5/00

(52) U.S. Cl. CPC A61B 5/02108; A61B 5/7221; A61B 5/7425; A61B 2562/0247

(57) ABSTRACT

Provided are an electronic device and an operation method of the same. The electronic device includes: a display panel including pixels that emit light; a light sensor disposed on the display panel and sensing external light; a pressure sensor sensing pressure applied from the outside; and a processor applied with a pressure signal sensed by the pressure sensor and a first pulse wave signal sensed by the light sensor, wherein the processor is configured to: analyze the first pulse wave signal in a blood pressure measurement mode and display a first indicator on the display panel when a first abnormal section is calculated in the first pulse wave signal, display a second indicator different from the first indicator on the display panel when the first abnormal section is not calculated, and calculate blood pressure information based on the first pulse wave signal and the pressure signal.

### CROSS-REFERENCE TO RELATED APPLICATION

This application claims priority under 35 U.S.C. 119 to Korean Patent Application No. 10-2022-0099642, filed on Aug. 10, 2022, in the Korean Intellectual Property Office, and all the benefits accruing therefrom, the entire contents of which are herein incorporated by reference.

### TECHNICAL FIELD

The disclosure relates to an electronic device and an operation method of the same.

### Discussion of the Related Art

A display device, which is a device for displaying a screen, is used not only in TVs and monitors, but also in portable smart phones and tablet PCs. In the case of a portable display device, for example, various functions such as a camera and a fingerprint sensor may be provided in the display device.

Meanwhile, as the healthcare industry is recently in the spotlight, methods for more conveniently acquiring biometric information about health are being developed. For example, there are attempts to replace the traditional oscillometric pulse measuring device with a portable electronic device. However, an electronic pulse measuring device can include an independent light source, sensor, and display, which can be inconvenient to carry separately.

### SUMMARY

Aspects of the disclosure provide an electronic device and an operation method of the same capable of inducing a pressure applied to a pressure sensor to increase to a predetermined level, and improving the accuracy of a blood pressure calculation by displaying a second indicator on a display panel.

However, aspects of the disclosure are not restricted to those set forth herein. The above and other aspects of the disclosure will become more apparent to one of ordinary skill in the art to which the disclosure pertains by referencing the detailed description of the disclosure given below.

According to an embodiment of the disclosure, an electronic device includes a display panel, a light sensor disposed on the display panel, a pressure sensor configured to sense an applied pressure, and a processor configured to receive a pressure signal from the pressure sensor and a first pulse wave signal from the light sensor, wherein the processor is configured to: analyze the pressure signal, display a first indicator on the display panel in response to a first abnormal section being calculated from the pressure signal, display a second indicator different from the first indicator on the display panel in response to the first abnormal section not being calculated, and calculate blood pressure information based on the first pulse wave signal and the pressure signal.

In various embodiments, the processor is configured to analyze the first pulse wave signal to display the first indicator on the display panel in response to a second abnormal section being calculated based on the first pulse wave signal.

In various embodiments, the processor is configured to calculate at least one measurement section as the first abnormal section in response to a magnitude of the pressure signal in the at least one measurement section among the measurement sections of the pressure signal does not exist within a first critical range.

In various embodiments, the first critical range may have a constant pressure width and increases with time to a preset degree, and the processor may be configured to display a guide indicator whose shape changes over time in response to the first critical range on the display panel.

In various embodiments, the processor may be configured to display the magnitude of the pressure signal on the guide indicator so that at least one of a shape, a color and a location changes over the time.

In various embodiments, the processor may be configured to calculate at least one measurement section as the second abnormal section when a magnitude of the first pulse wave signal in the at least one measurement section among measurement sections of the first pulse wave signal may not exist within a second critical range.

In various embodiments, the display panel may include a display area in which the pixels and the light sensor may be disposed, and a non-display area disposed at one side of the display area, and wherein at least one of the first indicator and the second indicator may have a shape extending along an edge of the display area.

In various embodiments, the first indicator and the second indicator may have the same shape.

In various embodiments, the processor may be configured to further calculate a third pulse wave signal including a pulse wave signal value according to pressure based on the first pulse wave signal and the pressure signal.

In various embodiments, the processor may be configured to, generate a peak detection signal based on an amplitude corresponding to a peak of each cycle of the third pulse wave signal, calculate a peak value of the peak detection signal and a pressure value corresponding to the peak value of the peak detection signal and calculate a diastolic blood pressure lower than the pressure value, a systolic blood pressure higher than the pressure value, and a mean blood pressure according to the pressure value, and display the diastolic blood pressure and the systolic blood pressure on the display panel.

In various embodiments, the processor may be configured to calculate the mean blood pressure as a pressure value corresponding to the peak value.

In various embodiments, the processor may be configured to, calculate a first pressure value smaller than the pressure value corresponding to 60% to 80% of the peak value and a second pressure value greater than the pressure value in the peak detection signal, and calculate the first pressure value as the diastolic blood pressure and calculate the second pressure value as the systolic blood pressure.

In various embodiments, one cycle of the third pulse wave signal may include a plurality of waveforms having different amplitudes, and a peak value of a first waveform among the plurality of waveforms is defined as a pulse wave contraction value, a peak value of a second waveform among the plurality of waveforms is defined as a reflected pulse wave value, and when the pulse wave contraction value is defined as Sp, the reflected pulse wave value is defined as Rp, and a reflected pulse wave ratio is defined as Ri, the processor is configured to calculate the reflected pulse wave ratio by.

In various embodiments, the reflected pulse wave ratio may include a first period in which the reflected pulse wave ratio fluctuates within a first range, a second period in which the reflected pulse wave ratio fluctuates within a second range, and a third period in which the reflected pulse wave ratio fluctuates within a third range, and a width of the first range and a width of the third range are smaller than a width of the second range.

In various embodiments, the processor may be configured to, analyze the reflected pulse wave ratio to detect a start point of time at which the second period starts, calculate a third pressure value corresponding to the first pulse wave signal at the start point of time of the second period, set the third pressure value as a diastolic blood pressure, calculate a fourth pressure value corresponding to the first pulse wave signal at a point of time at the third period starts after the second period, and calculate the fourth pressure value as a systolic blood pressure.

According to yet another aspect of the disclosure, a method of operating an electronic device, including a display panel, a light sensor, a pressure sensor, and a processor is provided. The method includes calculating a first abnormal section based on a pressure signal, displaying a first indicator on the display panel in response to the first abnormal section being calculated from the pressure signal, displaying the first indicator on the display panel in response to the first abnormal section being calculated from the pressure signal, displaying a second indicator different from the first indicator on the display panel in response to the first abnormal section not being calculated, and calculating blood pressure information based on the first pulse wave signal and the pressure signal.

In various embodiments, the first abnormal section may be calculated by analyzing the pressure signal, such that when the magnitude of the pressure signal in at least one measurement section among the measurement sections of the pressure signal may not exist within a first critical range, the at least one measurement section may be calculated as the first abnormal section.

An operation method of an electronic device may further in include displaying a guide indicator so that at least one of a shape, a color and a location changes over time in response to the first critical range on the display panel, wherein the first critical range has a constant pressure width and increases with time to a preset degree.

In various embodiments, the method of operating an electronic device may further include calculating a second abnormal section by analyzing a first pulse wave signal, wherein the second abnormal section may be calculated by analyzing the first pulse wave signal, such that when a magnitude of the first pulse wave signal in at least one measurement section among measurement sections of the first pulse wave signal does not exist within a second critical range, the at least one measurement section may be calculated as the second abnormal section.

In various embodiments, the display panel may include a display area in which the pixels and the light sensor are disposed, and a non-display area can be disposed at one side of the display area, where at least one of the first indicator and the second indicator has a shape extending along an edge of the display area.

According to the electronic device and the operation method of the same according to the various embodiments, a user's blood pressure may be measured by applying, by a user, a pressure to a pressure sensor, detecting light reflected from a blood vessel of a user's finger with a light sensor of a display panel, and analyzing the pulse wave signal and a pressure signal according to an amount of detected light.

In various embodiments, the pressure applied to the pressure sensor may be induced to increase to a certain level by displaying the guide indicator on the electronic device and the measured pressure signal on the display panel. In addition, when the abnormal measurement section is calculated from the pulse wave signal or the pressure signal, the accuracy of blood pressure calculation may be improved by displaying a second indicator on the display panel.

According to an embodiment of the disclosure, an electronic device includes, a display panel, a light sensor disposed on the display panel, a pressure sensor configured to sense an applied pressure, a processor configured to receive a pressure signal from the pressure sensor and a first pulse wave signal from the light sensor, wherein the processor is configured to: calculate blood pressure information based on the first pulse wave signal and the pressure signal, and determine that a magnitude of the pressure signal is not within a first critical range.

In various embodiments, the electronic device includes a driving unit configured to activate the pressure sensor.

In various embodiments, the electronic device driving unit is further configured to transmit a driving signal to the light sensor PS to activate the light sensor PS.

In various embodiments, the electronic device includes a touch sensor configured to detect a touch event, and an indicator generation unit configured to generate a guide indicator on the display panel DSP, wherein the guide indicator includes a pressure guide configured to indicate the applied pressure.

However, the embodiments of the present invention are not restricted to the features set forth herein. The above and other effects of the embodiments will become more apparent to one of daily skill in the art to which the embodiments pertain by referencing the claims.

### BRIEF DESCRIPTION OF THE DRAWINGS

The above and other aspects and features of the disclosure will become more apparent by describing in detail embodiments thereof with reference to the attached drawings, in which:

FIG. 1 is a perspective view of an electronic device, according to an embodiment;

FIG. 2 is a block diagram of the electronic device of FIG. 1, according to an embodiment;

FIG. 3 is a schematic cross-sectional view of the electronic device of FIG. 1, according to an embodiment;

FIG. 4 is a cross-sectional view of a display panel of the electronic device of FIG. 3, according to an embodiment;

FIG. 5 is a schematic layout view of a pressure sensor, according to an embodiment;

FIG. 6 is a cross-sectional view of the pressure sensor of FIG. 5, according to an embodiment;

FIG. 7 is a schematic layout view of a pressure sensor, according to an embodiment;

FIG. 8 is a cross-sectional view of the pressure sensor of FIG. 7, according to an embodiment;

FIG. 9 is a cross-sectional view of a pressure sensor, according to an embodiment;

FIG. 10 is a block diagram illustrating a control unit of an electronic device, according to an embodiment;

FIG. 11 is a flowchart illustrating an operation method of an electronic device, according to an embodiment;

FIG. 12 is a schematic diagram illustrating a pressure application step by a user, according to an embodiment;

FIG. 13 is a graph of a pressure signal representing a pressure measurement value versus time, according to an embodiment;

FIGS. 14 and 15 are graphs of a pulse wave signal representing a pulse wave measurement value versus time, according to an embodiment;

FIG. 16 is an enlarged graph of a first pulse wave signal, according to an embodiment;

FIG. 17 is a schematic view illustrating a display area of the electronic device, according to an embodiment;

FIG. 18 is an enlarged schematic view of a pressure guide, according to an embodiment;

FIG. 19 is a schematic view illustrating a display area of the electronic device, according to an embodiment;

FIGS. 20 and 21 are schematic views illustrating display areas of the electronic device, according to an embodiment;

FIG. 22 is a schematic view illustrating a display area of the electronic device, according to an embodiment;

FIG. 23 is a flowchart illustrating a method of calculating blood pressure, according to an embodiment;

FIG. 24 is a graph illustrating a waveform of a peak detection signal, according to an embodiment;

FIG. 25 is a flowchart illustrating a method of calculating blood pressure, according to an embodiment;

FIG. 26 is a graph illustrating a waveform of one cycle of a pulse wave signal, according to an embodiment; and

FIG. 27 is a graph illustrating a pulse wave signal and a reflected pulse wave ratio, according to an embodiment.

### DETAILED DESCRIPTION OF THE EMBODIMENTS

The various embodiments of the invention will now be described more fully hereinafter with reference to the accompanying drawings, in which the various embodiments of the invention are shown. This invention may, however, be embodied in different forms and should not be construed as limited to the embodiments set forth herein.

It will also be understood that when a layer is referred to as being “on” another layer or substrate, it can be directly on the other layer or substrate, or intervening layers may also be present. The same reference numbers indicate the same components throughout the specification.

It will be understood that, although the terms “first,” “second,” etc. may be used herein to describe various elements, these elements should not be limited by these terms. These terms are only used to distinguish one element from another element. For instance, a first element discussed below could be termed a second element without departing from the teachings of the invention. Similarly, the second element could also be termed the first element.

Hereinafter, specific embodiments will be described with reference to the accompanying drawings.

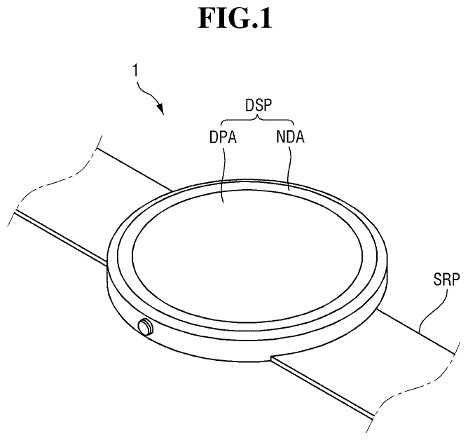

FIG. 1 is a perspective view of an electronic device according to an embodiment.

Referring to FIG. 1, an electronic device 1 according to an embodiment includes a display panel, DSP, where the display panel, DSP, can display a moving image or a still image. The display panel, DSP, may include a display area DPA in which an image may be presented and a non-display area NDA in which an image would not be presented.

Although the electronic device 1 including the display panel DSP is depicted in FIG. 1, as a smart watch, the disclosure is not limited thereto. For example, examples of the applicable electronic device 1 may include various wearable electronic devices including the smart watch, portable electronic devices such as a smart phone, a mobile phone, a tablet PC, a personal digital assistant (PDA), a portable multimedia player (PMP), a portable game machine, a notebook computer, a digital camera, a camcorder, and the like. Furthermore, a blood pressure measurement module may also be incorporated into a fixed or mobile device, including a display panel DSP, such as a personal computer monitor, a car navigation system, a car dashboard, an external advertising board, an electric sign, various medical devices, various inspection devices, a refrigerator, or a washing machine, which are intended to be included in the scope of application of the embodiments, even if such devices are not portable electronic devices. The various electronic devices 1 including the display panel DSP listed above may also be referred to as a display device.

The electronic device 1 of FIG. 1 may be configured to be worn on a portion of a body of a user (or a subject). For example, the electronic device 1 may be configured to be worn on a wrist or an ankle. To this end, the electronic device 1 may further include a strap SRP configured to fix the display panel DSP on a portion of the body of the user.

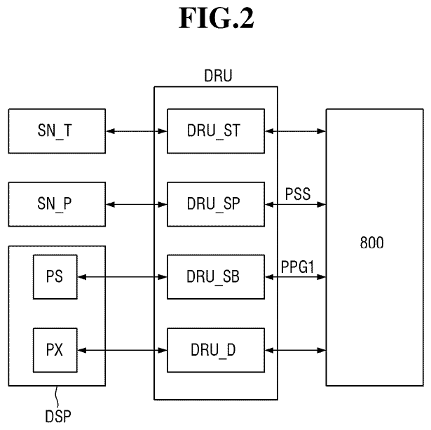

FIG. 2 is a block diagram of the electronic device of FIG. 1, according to an embodiment.

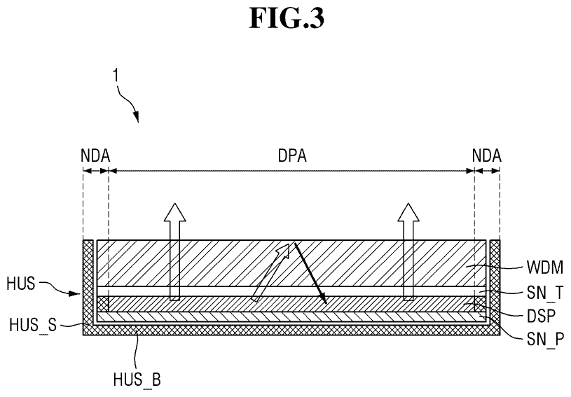

Referring to FIGS. 2 and 3, the electronic device 1 may further include a light sensor PS, a pressure sensor SN_P, a touch sensor SN_T, a driving unit DRU, and a processor 800 in addition to the display panel DSP. The display panel DSP may include a display area DPA, including a plurality of pixels PX, in which an image can be displayed. The display panel DSP may include a non-display area NDA in which an image would not be displayed.

In various embodiments, the pressure sensor SN_P may sense the magnitude of applied pressure, and the light sensor PS may sense the magnitude of light reflected from a user's blood vessel. In addition, the touch sensor SN_T may sense whether or not a touch event is input and the coordinates of the touch event.

In various embodiments, the driving unit DRU may include a display driving unit DRU_D, a light sensor driving unit DRU_SB, a pressure sensor driving unit DRU_SP, and a touch sensor driving unit DRU_ST.

In various embodiments, the display driving unit DRU_D may process image information received from the outside by the electronic device 1 or image information stored by the electronic device 1 to drive the display panel DSP so that the display panel DSP displays a corresponding image. In addition, the display driving unit DRU_D may process stored image information in response to a user's input or may generate and process new image information and provide the processed image information to the display panel DSP. In addition, the display driving unit DRU_D may process the stored or new image information based on information sensed by the light sensor PS, the pressure sensor SN_P, and the touch sensor SN_T to provide the processed image information to the display panel DSP. Furthermore, the display driving unit DRU_D may perform a function such as correcting an image processing signal by its own feedback circuit, where the function of the display driving unit DRU_D is not limited to that illustrated above.

In various embodiments, the light sensor driving unit DRU_SB, the pressure sensor driving unit DRU_SP, and the touch sensor driving unit DRU_ST may serve to drive operations of the sensors or process information sensed by the sensors. Although the functions of the sensors and the functions of the light sensor driving unit DRU_SB, the pressure sensor driving unit DRU_SP, and the touch sensor driving unit DRU_ST have been described separately, this is for convenience, and the functions of each sensor may also be performed by another driving unit, including the light sensor driving unit DRU_SB, the pressure sensor driving unit DRU_SP, and the touch sensor driving unit DRU_ST, and/or the display driving unit DRU_D.

In various embodiments, the light sensor driving unit DRU_SB, the pressure sensor driving unit DRU_SP, and the touch sensor driving unit DRU_ST may be provided for each sensor.

The pressure sensor driving unit DRU_SP may transmit a driving signal to the pressure sensor SN_P to activate the pressure sensor SN_P, and receive information measured by the pressure sensor SN_P to calculate the magnitude of pressure.

The light sensor driving unit DRU_SB may transmit a driving signal to the light sensor PS to activate the light sensor PS, and calculate the amount of light reflected by the user based on information measured by the light sensor PS.

The touch sensor driving unit DRU_ST may transmit a driving signal to the touch sensor SN T and calculate whether or not a touch event has occurred and the coordinates of the touch event based on information sensed by the touch sensor SN_T.

In various embodiments, the driving unit DRU may be provided in the form of a driving chip. Although each driving unit DRU may be provided in the form of a distinct driving chip, a plurality of driving units DRU may be integrated into one driving chip. In an embodiment, the electronic device 1 may include the display panel DSP, and the driving unit DRU may be mounted on the display panel DSP in the form of one or more driving chips.

In various embodiments, the processor 800 may be provided in the form of a driving chip. The processor 800 may receive a pressure measurement value according to time from the pressure sensor driving unit DRU_SP. In addition, the processor 800 may receive data on a critical range for determining an abnormal measurement section from a memory. In addition, the processor 800 may receive light sensing data according to time from the light sensor driving unit DRU_SB. In addition, the processor 800 may receive data regarding a critical range for determining an abnormal measurement section from a memory.

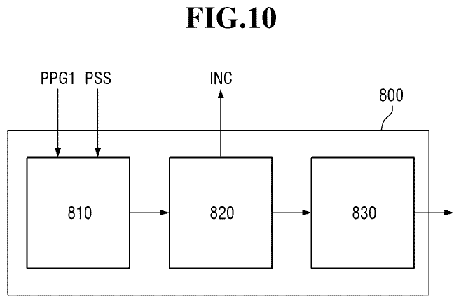

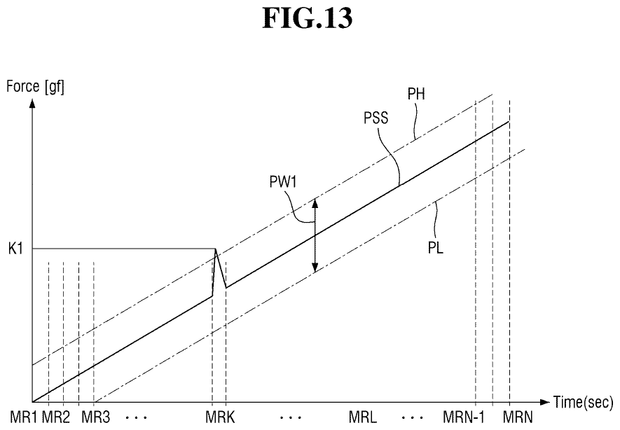

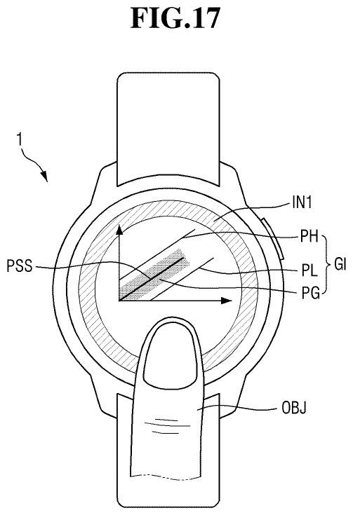

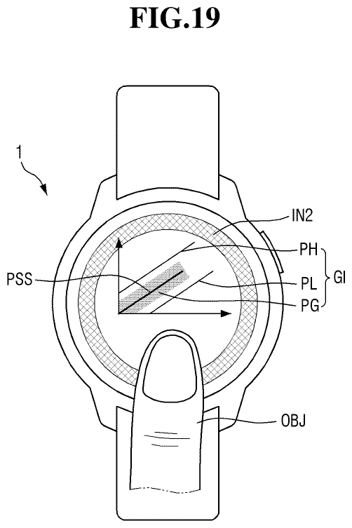

In various embodiments, the processor 800 may calculate an abnormal measurement section from a pressure signal (PSS in FIG. 13). In addition, the processor 800 may perform control, so that a guide indicator (GI in FIG. 17), a first indicator (IN1 in FIG. 17), and a second indicator (IN2 in FIG. 19) are displayed on the display panel DSP utilizing a display control signal INC, as shown in FIG. 10.

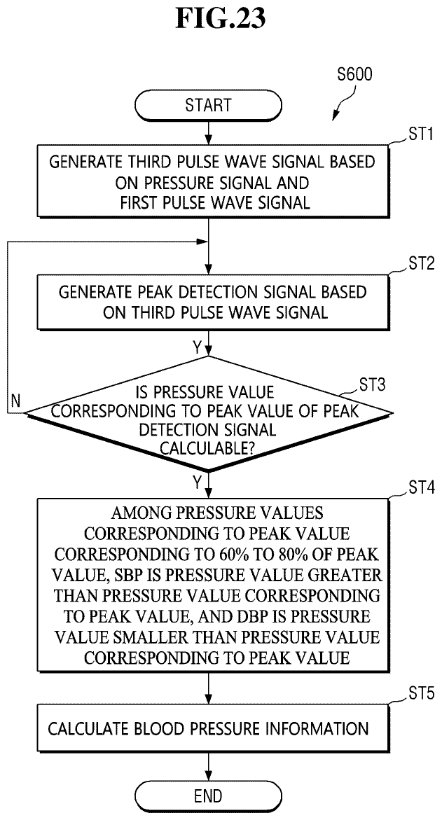

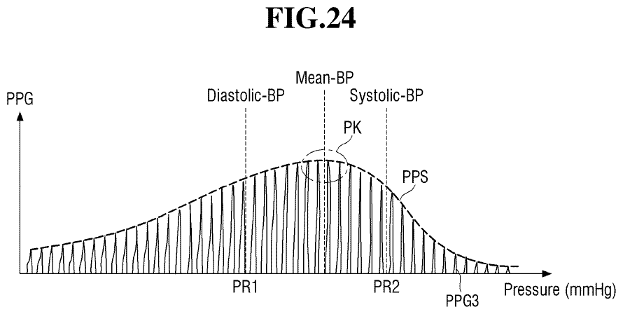

In addition, the processor 800 may generate a peak detection signal (PPS in FIG. 24) based on data on the period and amplitude of a pulse wave signal. The processor 800 may calculate a blood pressure based on a peak value of the generated peak detection signal PPS. This will be described later with reference to FIG. 23.

FIG. 3 is a schematic cross-sectional view of the electronic device of FIG. 1.

Referring to FIG. 3, the electronic device 1 may include a display panel DSP and a plurality of sensors. The plurality of sensors may include a light sensor PS, a pressure sensor SN_P, and a touch sensor SN_T. In addition, the electronic device 1 may further include an accommodation container HUS for accommodating the display panel DSP and the plurality of sensors, and a protective member WDM for protecting the display panel DSP.

In various embodiments, the display panel DSP can display a moving image or a still image. Examples of the display panel DSP may include a light receiving display panel such as a liquid crystal display panel (LCD) and an electrophoretic display panel (EPD), as well as a self-light emitting display panel such as an organic light emitting display panel (OLED), an inorganic light emitting display panel (inorganic EL), a quantum dot light emitting display panel (QED), micro LED display panel, a nano LED display panel, a plasma display panel (PDP), a field emission display panel (FED), and a cathode ray display panel (CRT). Hereinafter, the organic light emitting display panel (OLED) will be described as an example of the display panel DSP, and the organic light emitting display panel applied to the embodiment will be simply abbreviated as the display panel DSP unless a special distinction is involved. However, the embodiment is not limited to the organic light emitting display device, and other display devices listed above or known in the technical field may be applied within the scope of the inventive concept.

In various embodiments, the display panel DSP emits light emitted from the light emitting layer to the outside to display an image. The display panel DSP can include a first surface (i.e., a front surface) and a second surface (i.e., a rear surface) opposite the first surface. The display panel DSP may be designed to emit the light from the light emitting layer to the first surface and/or the second surface. It is illustrated in the drawing that the display panel DSP is a top emission type display panel that emits light through the first surface, that is, upwardly emits light. However, the disclosure is not limited thereto, and the display panel DSP is applicable as a bottom emission type display panel that emits light through the second surface or a double-sided emission type display panel that emits light through both the first surface and the second surface.

In various embodiments, a planar shape of the display panel DSP may be a circular shape or a shape including a portion of the circular shape as illustrated in FIG. 1, but is not limited thereto. The planar shape of the display panel DSP may be, for example, a polygon such as a square, a rectangle, a hexagon, or an octagon. In addition, the planar shape of the display panel DSP may also be a polygon in which corner portions are inclined or curved.

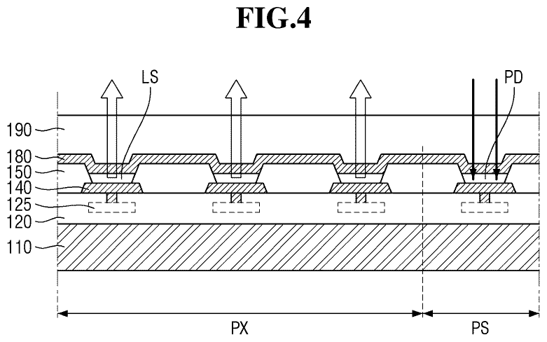

In various embodiments, the display panel DSP may include a display area DPA in which a display is performed and a non-display area NDA in which a display is not performed. The display area DPA may include a plurality of pixels (see ‘PX’ in FIG. 4). The pixels may serve to provide inspection light toward an object OBJ, where the wavelength of the inspection light may be an infrared wavelength, a visible light wavelength, a red wavelength of visible light, a green wavelength of visible light, a blue wavelength of visible light, and the like, which may be applied. The pixel PX may include at least one of, for example, a light emitting diode (LED), an organic light emitting diode (OLED), a laser diode (LD), a quantum dot (QD), a phosphor, or natural light.

In various embodiments, the display panel DSP may include a light sensor (PS in FIG. 4). The light sensor PS may include a photoelectric conversion layer (PD in FIG. 4) that receives light reflected or scattered from an object OBJ. The photoelectric conversion layer PD may include, for example, a photo diode, a photo transistor, a CMOS sensor, or CCD image sensor, and the like. A photo-plethysmography sensor may be configured to generate a pulse wave signal PPG by analyzing the amount of light received through the photoelectric conversion layer PD.

In various embodiments, a portion of the body of a user may be used for measuring blood pressure can involve pressure application and/or contact through the protective member WDM as illustrated in FIG. 3. The inspection light emitted from the display panel DSP may reach a portion of the body of the user, and may be reflected inside a subcutaneous tissue, where the reflected light may pass through a light-transmitting area, and may be incident on the photoelectric conversion layer (PD in FIG. 4).

In various embodiments, the non-display area NDA may not include pixels PX or may include dummy pixels. The non-display area NDA may be disposed along a peripheral portion of the display panel DSP, where a non-display area can be disposed at one side of the display area. In an embodiment, the non-display area NDA may surround an outer side surface of the display panel DSP, where the NDA can have a closed curve shape. The non-display area NDA may be recognized as a bezel area.

In some embodiments, the non-display area NDA may also be disposed inside the display area DPA. For example, the non-display area NDA positioned around the display area DPA may be recessed into the display area DPA. As another example, an island-shaped non-display area NDA completely surrounded by the display area DPA may be further positioned inside the display area DPA.

The plurality of sensors may include a pressure sensor SN_P, a light sensor PS, and a touch sensor SN_T.

The pressure sensor SN_P serves to sense the magnitude of input pressure. The pressure sensor SN_P may include, for example, a force sensor, a strain gauge, a gap capacitor, and the like, but is not limited thereto. A detailed description of the applicable pressure sensor SN_P will be described later.

The pressure sensor SN_P may be configured to generate a pressure signal PSS corresponding to the magnitude of the input pressure over time. To generate the pressure signal PSS, the pressure sensor SN_P may include a pressure signal generating unit. As another example, a portion or all of the pressure signal generating unit involved in generating the pressure signal PSS may also be installed in the light sensor driving unit DRU_SB, the pressure sensor driving unit DRU_SP, or the touch sensor driving unit DRU_ST.

The pressure sensor SN_P may be disposed on a lower portion of the display panel DSP, that is, on a second surface of the display panel DSP. The pressure sensor SN_P may overlap the second surface of the display panel DSP in a thickness direction. The pressure sensor SN_P may overlap the entire second surface of the display panel DSP or overlap a portion of the second surface of the display panel DSP.

In various embodiments, the pressure sensor SN_P may overlap the display area DPA of the display panel DSP. In an embodiment, the pressure sensor SN_P may overlap the non-display area NDA of the display panel DSP. In some embodiments, the pressure sensor SN_P may overlap both the display area DPA and the non-display area NDA.

In various embodiments, the pressure sensor SN_P may be attached onto the second surface of the display panel DSP. In this case, an adhesive member may be interposed between the pressure sensor SN_P and the second surface of the display panel DSP.

In various embodiments, the pressure sensor SN_P may be disposed on an upper portion of the display panel DSP, that is, on a first surface of the display panel DSP. The touch sensor SN_T may be referred to as a touch member.

In various embodiments, the touch sensor SN_T may be provided integrally with the display panel DSP. For example, the touch sensor SN_T may be formed on an encapsulation layer covering the light emitting element of the display panel DSP. As another example, the touch sensor SN_T may be provided as a distinct panel from the display panel DSP and may be attached onto the display panel DSP through a transparent bonding layer.

In various embodiments, the protective member WDM may be disposed on the touch sensor SN_T. The protective member WDM may include a transparent material. The protective member WDM may include, for example, glass, thin film glass, or ultra-thin glass, or a transparent polymer such as transparent polyimide. The protective member WDM may be referred to as a window or window member.

In various embodiments, the transparent bonding layer for bonding the touch sensor SN_T and the protective member WDM may be disposed between the touch sensor SN_T and the protective member WDM.

In various embodiments, the accommodation container HUS serves as a housing for accommodating the display panel DSP, the light sensor PS, the pressure sensor SN_P, the touch sensor SN_T, the driving unit DRU, the protective member WDM, and the like. The accommodation container HUS may include a bottom portion HUS_B and a sidewall portion HUS_S extending in a vertical direction from the bottom portion HUS_B. The display panel DSP, the light sensor PS, the pressure sensor SN_P, the touch sensor SN_T, the protective member WDM, and the like described above may be disposed in a space provided by the bottom portion HUS_B and the sidewall portion HUS_S.

FIG. 4 is a cross-sectional view of a display panel of the electronic device of FIG. 3, according to an embodiment.

Referring to FIG. 4, the display panel DSP may include a pixel PX and a light sensor PS.

More specifically, a circuit layer 120 is disposed on a substrate 110. The circuit layer 120 may include a pixel circuit 125. The pixel circuit 125 may include one or more transistors.

In various embodiments, a first electrode 140 may be disposed on the circuit layer for each pixel. A pixel defining film 150 may be disposed on the first electrode 140 to partition each pixel. An active layer may be disposed on the first electrode 140 exposed by the pixel defining film 150. A second electrode 180 may be disposed on the active layer. The first electrode 140 may be a pixel electrode provided for each of the pixel(s) PX and the light sensor PS, and the second electrode 180 may be a common electrode integrally connected regardless of the pixel PX and the light sensor PS, but the disclosure is not limited thereto. An encapsulation layer 190 may be disposed on the second electrode 180. A touch layer may be further disposed on the encapsulation layer 190.

The active layer of the pixel PX may include a light emitting layer LS . The active layer of the light sensor PS may include a photoelectric conversion layer PD. The light emitting layers LS of at least some of the pixels PX may serve as light sources. That is, light emitted from the light emitting layers LS of at least some of the pixels PX may be used as the inspection light for measuring blood pressure. In addition, the light emitting layers LS of at least some of the pixels PX may simultaneously perform a screen display function and a light source function for measuring a pulse wave. The photoelectric conversion layer PD of the light sensor PS may receive reflected light reflected from the light source.

In various embodiments, the light emitting layer LS of the pixel PX and the photoelectric conversion layer PD of the light sensor PS may include a hole injection layer and/or a hole transporting layer on a lower side of the light emitting layer/photoelectric conversion layer, and may further include an electron transporting layer and/or an electron injection layer on an upper side of the light emitting layer/photoelectric conversion layer. In various embodiments, the hole injection layer, the hole transporting layer, the electron transporting layer, and the electron injection layer may be applied as the same material layer without distinction between the pixel PX and the light sensor PS. Furthermore, the hole injection layer, the hole transporting layer, the electron transporting layer, and the electron injection layer may also be provided as a common layer integrally connected without distinction between the pixels PX.

In various embodiments, the display panel DSP, the pixel PX, and the light sensor PS can share a plurality of layers. Therefore, the light sensor PS may be internalized in the display panel DSP with a simple structure.

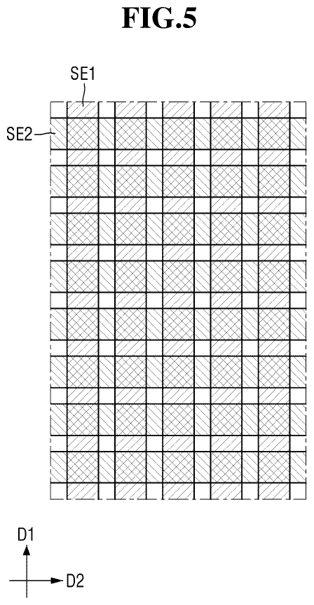

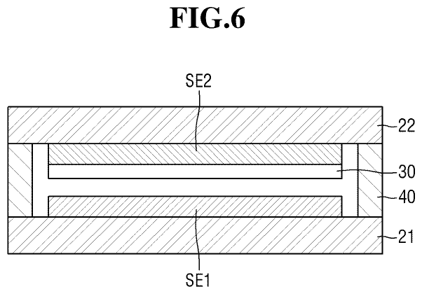

FIG. 5 is a schematic layout view of a pressure sensor according to an embodiment. FIG. 6 is a cross-sectional view of the pressure sensor of FIG. 5. FIGS. 5 and 6 exemplarily illustrate a structure of a force sensor that is an example of the pressure sensor SN_P.

Referring to FIGS. 5 and 6, the pressure sensor SN_P may include a first electrode SE1, a second electrode SE2, and a pressure sensing layer 30 disposed between the first electrode SE1 and the second electrode SE2.

Each of the first electrode SE1 and the second electrode SE2 may include a conductive material. In various embodiments, the first electrode SE1 and the second electrode SE2 may be formed of a metal, such as silver (Ag) or copper (Cu), a transparent conductive oxide such as ITO, IZO, or ZIO, carbon nanotubes, a conductive polymer, or the like. One of the first electrode SE1 and the second electrode SE2 may be a driving electrode, and the other may be a sensing electrode.

In various embodiments, the pressure sensing layer 30 may include a pressure sensitive material. The pressure sensitive material may include carbon or metal nanoparticles such as nickel, aluminum, tin, and copper. The pressure sensitive material may be disposed within a polymer resin in the form of particles, but is not limited thereto. In the pressure sensing layer 30, the pressure-sensitive material can have an electrical resistance that decreases as the pressure increases. Here, by measuring the electrical resistance of the pressure sensing layer 30 through the first electrode SE1 and the second electrode SE2, it is possible to sense whether or not a pressure is applied and the magnitude of the pressure. The pressure sensing layer 30 may be transparent or opaque.

In various embodiments, each of the first electrode SE1 and the second electrode SE2 may be arranged in a line. For example, a plurality of first electrodes SE1 may extend in parallel in a first direction D1, and a plurality of second electrodes SE2 may extend in a second direction D2 intersecting the first direction D1, for example, perpendicular to the first direction D1. The plurality of first electrodes SE1 and the plurality of second electrodes SE2 can have a plurality of overlapping areas at mutually intersecting portions. Each overlapping area may have a matrix arrangement. Each overlapping area may be a pressure sensing cell. That is, the pressure sensing layer 30 may be disposed in each overlapping area to perform pressure sensing at a corresponding position.

In various embodiments, the pressure sensor SN_P may include two sensor substrates facing each other, as shown in FIG. 6. Each sensor substrate may include a base material 21, 22. A first base material 21 of the first sensor substrate and a second base material 22 of the second sensor substrate may include polyethylene, polyimide, polycarbonate, polysulfone, polyacrylate, polystyrene, polyvinyl chloride, polyvinyl alcohol, poly norbornene, and polyester-based materials, respectively. In an embodiment, the first base material 21 and the second base material 22 may be formed of a polyethylene terephthalate (PET) film or a polyimide film.

In various embodiments, the first electrode SE1, the second electrode SE2, and the pressure sensing layer 30 may be included in the first sensor substrate or the second sensor substrate. For example, the first electrode SE1 and the pressure sensing layer 30 may be included in the first sensor substrate, and the second electrode SE2 may be included in the second sensor substrate. The first electrode SE1 may be disposed on one surface of the first base material 21 facing the second base material 22. The second electrode SE2 may be disposed on one surface of the second base material 22 facing the first base material 21, and the pressure sensing layer 30 may be disposed on the second electrode SE2. The first sensor substrate and the second sensor substrate may be bonded to each other by a bonding layer 40. The bonding layer 40 may be disposed along an edge of each sensor substrate, but is not limited thereto.

In various embodiments, the first electrode SE1, the second electrode SE2, and the pressure sensing layer 30 may also be included in one sensor substrate. For example, the first electrode SE1 may be disposed on one surface of the first base material 21, the pressure sensing layer 30 may be disposed on the first electrode SE1, and the second electrode SE2 may be disposed on the pressure sensing layer 30.

In various embodiments, the pressure sensor SN_P including the above-described force sensor may be transparent or opaque. In the case of the transparent pressure sensor SN_P, the first base material 21 and the second base material 22 may be formed of a transparent material, the first electrode SE1 and the second electrode SE2 may be formed of a transparent conductive material, and the pressure sensing layer 30 may also be formed of a transparent material. In the case of the opaque pressure sensor SN_P, an electrode or a pressure sensitive material may be selected from various materials regardless of whether it is transparent or not.

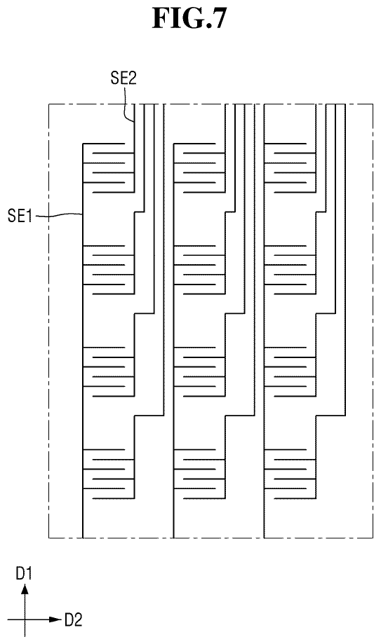

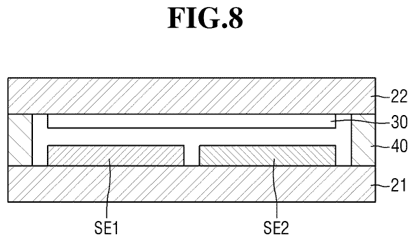

FIG. 7 is a schematic layout view of a pressure sensor according to an embodiment. FIG. 8 is a cross-sectional view of the pressure sensor of FIG. 7. FIGS. 7 and 8 exemplarily illustrate another structure of the force sensor.

Referring to FIGS. 7 and 8, the pressure sensor SN_P according to the embodiment is different from the embodiment of FIGS. 5 and 6 in that the first electrode SE1 and the second electrode SE2 are disposed on the same layer. Specifically, for example, the first electrode SE1 and the second electrode SE2 are disposed on one surface of the first base material 21. The first electrode SE1 and the second electrode SE2 are disposed to be adjacent to each other. The first electrode SE1 and the second electrode SE2 may each include a plurality of branch portions, and may have a comb electrode shape in which the branch portions are disposed to cross each other. The pressure sensing layer 30 is formed on the second base material 22 and disposed on the first electrode SE1 and the second electrode SE2.

In various embodiments, the first electrode SE1 and the second electrode SE2 do not overlap each other in the thickness direction, but are disposed to be adjacent to each other in plan view. When pressure is applied, a current may flow between the first and second electrodes SE1 and SE2 adjacent to each other through the pressure sensing layer 30. Such a structure may be advantageous for measuring shear pressure.

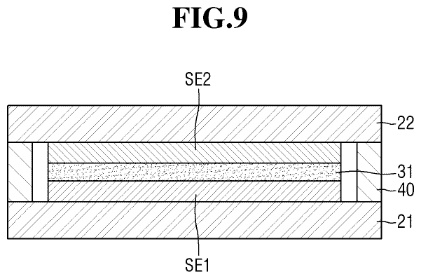

FIG. 9 is a cross-sectional view of a pressure sensor according to an embodiment. FIG. 9 illustrates a gap capacitor as an example of the pressure sensor SN_P.

Referring to FIG. 9, the pressure sensor SN_P according to the embodiment may include a first electrode SE1, a second electrode SE2, and a dielectric constant modifying material layer 31 disposed between the first electrode SE1 and the second electrode SE2. The pressure sensor SN_P according to the embodiment may have substantially the same structure as the pressure sensor SN_P according to the embodiments of FIGS. 5 and 6 except that the dielectric constant modifying material layer 31 is disposed between the first electrode SE1 and the second electrode SE2 instead of the pressure sensing layer 30.

In various embodiments, the dielectric constant modifying material layer 31 is a material whose dielectric constant is changed according to applied pressure, and various materials known in the art may be applied. Since the dielectric constant of the dielectric constant modifying material layer 31 varies according to the applied pressure, the magnitude of the applied pressure may be measured by measuring a capacitance value between the first electrode SE1 and the second electrode SE2.

The pressure sensor SN_P including the above-described gap capacitor may be transparent or opaque. In the case of the transparent pressure sensor SN_P, the first electrode SE1 and the second electrode SE2 may be formed of a transparent conductive material, and the dielectric constant modifying material layer 31 may also be formed of a transparent material. In the case of the opaque pressure sensor SN_P, an electrode or a pressure sensitive material may be selected from various materials regardless of whether it is transparent or not.

FIG. 10 is a block diagram illustrating a control unit of an electronic device according to an embodiment.

In various embodiments, the processor 800 includes a measurement section calculation unit 810, an indicator generation unit 820, and a blood pressure calculation unit 830.

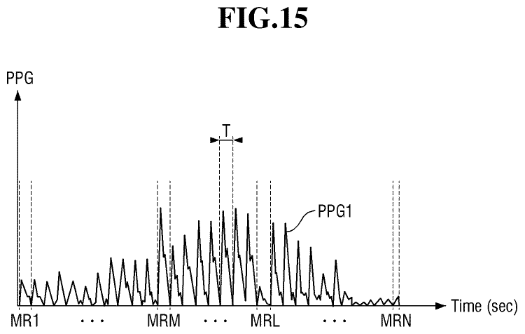

In various embodiments, the measurement section calculation unit 810 may receive a pressure signal (PSS in FIG. 13) having a pressure measurement value according to time from the pressure sensor driving unit DRU_SP. In addition, the measurement section calculation unit 810 may receive data on a critical range for determining an abnormal measurement section from the memory. In addition, the measurement section calculation unit 810 may receive a first pulse wave signal (PPG1 in FIG. 15) including light sensing data according to time from the light sensor driving unit DRU_SB. In addition, the measurement section calculation unit 810 may receive data on a critical range for determining an abnormal measurement section from the memory.

Accordingly, the measurement section calculation unit 810 may calculate an abnormal measurement section from the pressure signal PSS. For example, the measurement section calculation unit 810 may determine whether the pressure signal PSS of at least one measurement section among a plurality of measurement sections of the pressure signal PSS is an abnormal measurement section (e.g., a K-th measurement section MRK). The measurement section calculation unit 810 may output the calculated abnormal measurement section (e.g., the K-th measurement section MRK) to the indicator generation unit 820.

In various embodiments, the indicator generation unit 820 may receive the pressure signal PSS and the first pulse wave signal from the measurement section calculation unit 810, where the processor can be configured analyze the pressure signal to display a first indicator on the display panel in response to a second abnormal section being calculated based on the pressure signal. The processor can also be configured to calculate at least one measurement section as the second abnormal section in response to a magnitude of the pressure signal in the at least one measurement section among the measurement sections of the pressure signal does not exist within a first critical range.

In various embodiments, the indicator generation unit 820 may generate a display control signal INC to display a guide indicator for measuring pressure on the display panel DSP. In addition, the indicator generation unit 820 may perform control so that the received pressure signal PSS is displayed on the display panel DSP. In addition, the indicator generation unit 820 may perform control so that a first indicator (IN1 in FIG. 17) or a second indicator (IN2 in FIG. 19) is displayed on the display panel DSP depending on the presence or absence of the abnormal measurement section in the received pressure signal PSS and first pulse wave signal PPG1. That is, the indicator generation unit 820 may perform control so that the guide indicator (GI in FIG. 17), the first indicator (IN1 in FIG. 17), and the second indicator (IN2 in FIG. 17) are displayed on the display panel DSP through the display control signal INC. The indicator generation unit 820 may output the pressure signal PSS and the first pulse wave signal PPG1 to the blood pressure calculation unit 830.

In various embodiments, the blood pressure calculation unit 830 may generate a peak detection signal (PPS in FIG. 24) based on data on the period and amplitude of the pulse wave signal PPG. The blood pressure calculation unit 830 may calculate a blood pressure based on a peak value of the generated peak detection signal PPS. This will be described later with reference to FIG. 23.

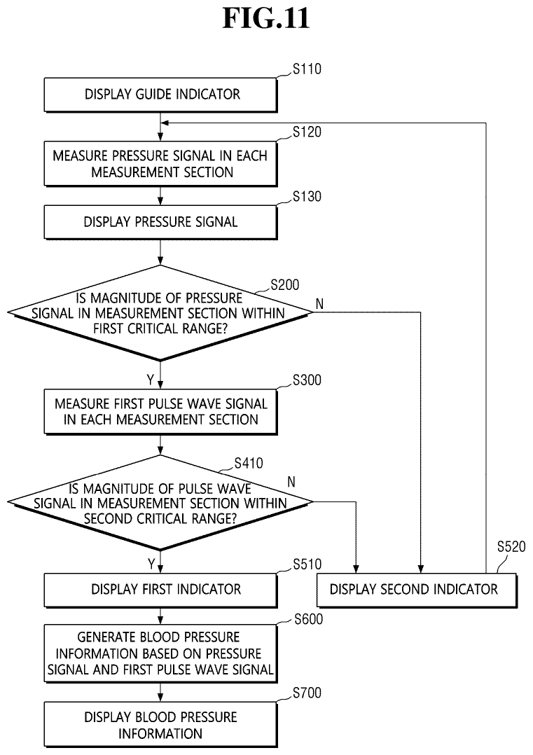

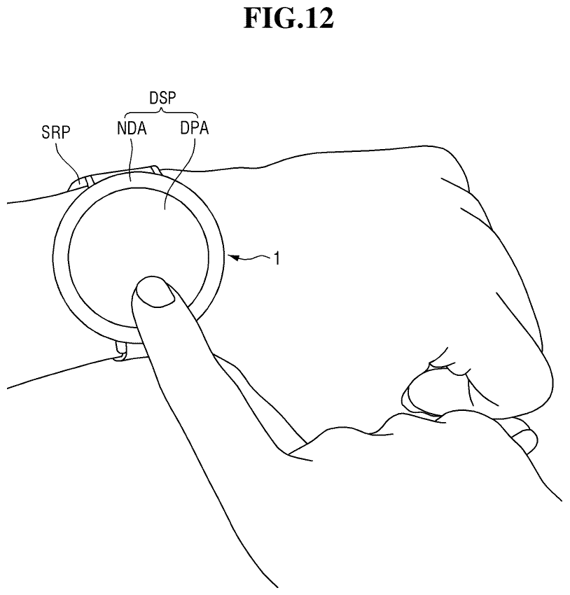

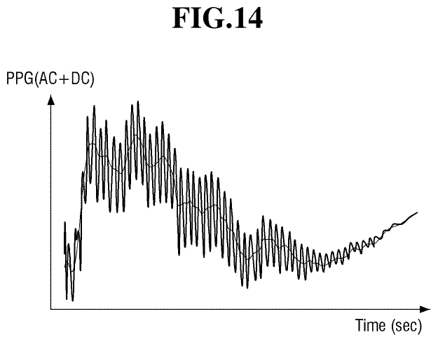

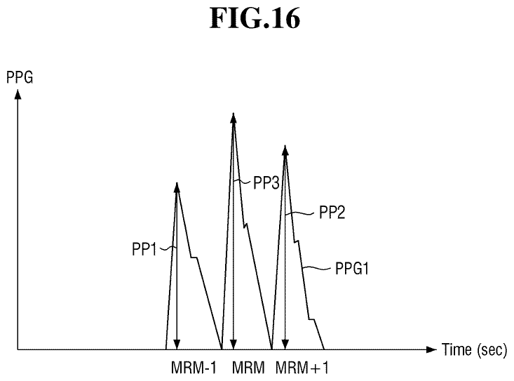

FIG. 11 is a flowchart illustrating an operation method of an electronic device according to an embodiment. FIG. 12 is a schematic diagram illustrating a pressure application step by a user. FIG. 13 is a graph of a pressure signal representing a pressure measurement value versus time. FIGS. 14 and 15 are graphs of a pulse wave signal representing a pulse wave measurement value versus time. FIG. 16 is an enlarged graph of a first pulse wave signal. Hereinafter, a method of measuring blood pressure of the electronic device 1 will be described with reference to FIGS. 7 to 11.

Referring to FIG. 11, first, the processor 800 displays the guide indicator GI on the display panel DSP (S110).

Referring further to FIG. 12, in a blood pressure measurement mode, the user may apply pressure in various methods through which the pressure sensor SN_P of the electronic device 1 may recognize a pressing force. For example, as illustrated in FIG. 12, the user may press the upper surface (protective member WMD) of the electronic device 1 by using a finger, other body portions, or other external devices on the front surface of the electronic device 1 in a state in which the electronic device 1 is worn on the wrist. In addition, the user may apply pressure by tightening a strap SRP attached to the electronic device 1, and the pressing method is not limited to those illustrated. The magnitude of the pressure applied to the upper surface of the electronic device 1 may be measured by the pressure sensor SN_P inside the electronic device 1.

In various embodiments, the processor 800 may display the guide indicator (GI in FIG. 17) on the display panel DSP in the blood pressure measurement mode. The guide indicator (GI in FIG. 17) may include information on upper limit pressure PH, lower limit pressure PL, and a first critical range PW1. This will be described later.

In various embodiments, the pressure sensor SN_P can measure the pressure signal PSS in each measurement section (S120).

Referring further to FIG. 13, the user may apply pressure to a position where the pressure sensor SN_P is disposed, and the disposed pressure sensor SN_P may measure a pressure measurement value applied by the user. A method of generating a pulse wave signal will be described in detail. For example, in a process in which the user brings his or her finger into contact with the electronic device 1, the pressure measurement value measured by the pressure sensor SN_P may gradually increase over time to reach a maximum value. As the pressure measurement value (i.e., contact pressure) increases, a blood vessel may constrict and a blood flow rate may decrease or become zero.

In various embodiments, the processor 800 may receive a pressure signal PSS having pressure measurement values according to a first to an N-th measurement section MR1 to MRN measured by the pressure sensor SN_P. Here, each of the first to N-th measurement sections MR1 to MRN may be a subdivision section for generating one cycle of the first pulse wave signal PPG1. Each of the first to N-th measurement sections MR1 to MRN may have a predetermined interval. For example, when any one of the first to N-th measurement sections MR1 to MRN is defined as a K-th measurement section MRK, the predetermined interval of the K-th measurement section MRK may be an interval having one cycle of the first pulse wave signal PPG1. However, the disclosure is not limited thereto, and any one measurement section may also have a larger interval or a smaller interval.

The magnitude of the pressure signal PSS received by the processor 800 may have different values in each of the first to N-th measurement sections MR1 to MRN. The magnitude of the pressure signal PSS may gradually increase in each of the first to N-th measurement sections MR1 to MRN. For example, as illustrated in FIG. 13, the pressure signal PSS measured in the K-th measurement section MRK may have a first pressure value K1.

In various embodiments, the pressure signal PSS sensed by the pressure sensor SN_P, the user needs to apply gradually increasing pressure to the pressure sensor SN_P. Therefore, it is very difficult for the user to continuously apply constantly increasing pressure to the pressure sensor SN_P throughout the first to N-th measurement sections MR1 to MRN in which the user measures the blood pressure. In this case, when the user applies pressure out of the critical range in at least one measurement section, inaccurate pressure signal PSS and pulse wave signal may be generated.

In various embodiments, the processor 800 displays the pressure signal PSS on the display panel DSP (S130). When the pressure signal PSS is sensed in each of the measurement sections, the processor 800 may display the sensed pressure signal PSS on the display panel DSP in real time. The pressure signal PSS displayed on the display panel DSP will be described later with reference to FIGS. 17 to 19.

In various embodiments, the processor 800 determines whether or not the magnitude of the pressure signal PSS in the measurement section is within the first critical range PW1 (S200).

In various embodiments, the first critical range PW1 may have a value between the upper limit pressure PH and the lower limit pressure PL, where a width of the first critical range PW1 may be a pressure value between the upper limit pressure PH and the lower limit pressure PL. In addition, the upper limit pressure PH, the lower limit pressure PL, and the first critical range PW1 may gradually increase in the first to N-th measurement sections MR1 to MRN. For example, the upper limit pressure in the K-th measurement section MRK may be smaller than the upper limit pressure in the N-th measurement section MRN, which is the last measurement section.

In various embodiments, the processor 800 may compare a size of any one of the first to N-th measurement sections MR1 to MRN with the first critical range PW1. For example, as illustrated in FIG. 13, when the pressure signal PSS of the K-th measurement section MRK has a first pressure value K1, the first pressure value K1 is greater than the upper limit pressure PH. That is, the first pressure value K1 does not exist within the first critical range PW1. Therefore, when the size of any one of the first to N-th measurement sections MR1 to MRN does not exist in the first critical range PW1, the processor 800 may calculate the K-th measurement section MRK as an abnormal measurement section (S140). Accordingly, if an abnormal section is detected in the pressure signal PSS (N in S200), the processor 800 can display a second indicator (IN2 in FIG. 19) on the display panel DSP (S520). The second indicator (IN2 in FIG. 19) will be described later with reference to FIGS. 17 to 19.

In various embodiments, the pressure signal PSS is within the first critical range PW1 in each of the first to N-th measurement sections MR1 to MRN, the processor 800 may recognize the pressure signal PSS as a normal pressure signal PSS.

In various embodiments, the light sensor measures a first pulse wave signal PPG1 in each measurement section (S300).

Referring further to FIGS. 14 and 15, in order to generate the first pulse wave signal PPG1, pulse wave information according to time is also utilized along with pressure data. During systole of a heart, a blood discharged from a left ventricle of the heart moves to peripheral tissues, and a blood volume in an arterial side increases. In addition, during the systole of the heart, red blood cells carry more oxygenated hemoglobin to the peripheral tissues. During diastole of the heart, there is partial suction of the blood from peripheral tissues towards the heart. In this case, when a peripheral blood vessel is irradiated with the light emitted from the display pixel, the irradiated light may be absorbed by the peripheral tissues. Light absorbance is dependent on hematocrit and blood volume. The light absorbance may have a maximum value in the systole of the heart and a minimum value in the diastole of the heart. Since the light absorbance is in inverse proportion to the amount of light incident on the light sensor PS, it is possible to estimate the light absorbance at a corresponding point of time through light reception data of the amount of light incident on the light sensor PS, and through this, as illustrated in FIG. 14, a pulse wave signal value according to time may be generated.

The pulse wave information according to time reflects the maximum value of light absorbance during the systole of the heart and the minimum value of the light absorbance during the diastole of the heart. In addition, the pulse wave information represents a phenomenon that vibrates according to a heartbeat cycle T. Therefore, the pulse wave information may reflect a change in blood pressure according to a heartbeat. Therefore, the light sensor driving unit DRU_SB may measure a value of the first pulse wave signal PPG1 according to a pressing time. However, the pulse wave signal PPG may include both an alternating current (AC) component and a direct current (DC) component. The processor 800 may generate the first pulse wave signal (PPG1 in FIG. 15) by removing the DC component from the pulse wave signal and plotting the pulse wave signal according to the magnitude of time.

In various embodiments, the processor 800 may receive the generated first pulse wave signal PPG1 from the light sensor driving unit DRU_SB. The processor 800 may receive the first pulse wave signals PPG1 generated sequentially according to the first to N-th measurement sections MR1 to MRN. For example, when any one of the first to N-th measurement sections MR1 to MRN is defined as an M-th measurement section MRM, a previous measurement section adjacent to the M-th measurement section MRM is defined as an M−1-th measurement section MRM−1, and a next measurement section adjacent to the M-th measurement section MRM is defined as an M+1-th measurement section MRM+1. Accordingly, the first pulse wave signal PPG1 may be sequentially generated in the M−1-th measurement section MRM−1, the M-th measurement section MRM, and the M+1-th measurement section MRM+1.

In various embodiments, the processor 800 determines whether or not the magnitude of the first pulse wave signal PPG1 in any one measurement section is within a second critical range (S410). The processor is configured to calculate at least one measurement section as the first abnormal section when a magnitude of the first pulse wave signal in the at least one measurement section among measurement sections of the first pulse wave signal is not within a second critical range.

Referring further to FIGS. 15 and 16, in order to determine the abnormal measurement section of the first pulse wave signal PPG1 in the M-th measurement section MRM, the processor 800 may calculate an amplitude in each of the first to N-th measurement sections MR1 to MRN. For example, as illustrated in FIG. 16, the processor 800 may calculate a first amplitude PP1 of the M−1-th measurement section MRK-1 of the first pulse wave signal PPG1. For example, the M−1-th measurement section MRM−1 of the first pulse wave signal PPG1 may be one cycle of the first pulse wave signal PPG1. In this case, the first amplitude PP1 of the M−1-th measurement section MRM−1 of the first pulse wave signal PPG1 may be a maximum value of a pulse wave waveform corresponding to the M−1-th measurement section MRM−1 of the first pulse wave signal. Alternatively, the first amplitude PP1 may be a peak value of a pulse wave waveform corresponding to the M−1-th measurement section MRM−1 of the first pulse wave signal. In addition, as another example, even when the pulse wave waveform corresponding to the M−1-th measurement section MRM−1 of the first pulse wave signal includes a plurality of peaks, the first amplitude PP1 may be the maximum value of the pulse wave waveform corresponding to the M−1-th measurement section MRM−1 of the first pulse wave signal.

In various embodiments, the processor 800 may calculate a third amplitude PP3 of the M-th measurement section MRM of the first pulse wave signal PPG1 and a second amplitude PP2 of the M+1-th measurement section MRM+1 thereof. A method in which the processor 800 calculates the third amplitude PP3 of the M-th measurement section MRM of the first pulse wave signal PPG1 and the second amplitude PP2 of the M+1-th measurement section MRM+1 thereof is substantially the same as the method in which the processor 800 calculates the first amplitude PP1 of the M−1-th measurement section MRM−1 of the first pulse wave signal PPG1, and thus a description thereof will be omitted.

In various embodiments, the processor 800 may determine whether or not the first pulse wave signal PPG1 of the M-th measurement section MRM is in the second critical range based on the calculated first amplitude PP1 and second amplitude PP2. For example, as illustrated in FIG. 16, the processor 800 may set the second critical range to determine whether or not the M-th measurement section MRM of the first pulse wave signal PPG1 is an abnormal measurement section. The pulse wave signal will be described in detail. As pressure is applied to the user's blood vessel, a blood vessel wall contracts, and accordingly, the pulse wave signal has a value proportional to the amount of blood inside the blood vessel. Therefore, when constantly increasing pressure is applied to the user's blood vessel, the pulse wave signal has a gradually increasing or decreasing waveform. That is, when a peak value of one cycle of the pulse wave signal does not gradually increase or decrease, accurate blood pressure information may not be calculated. Therefore, the processor 800 may determine the corresponding section as the abnormal measurement section. Accordingly, the second critical range may be a range between the first amplitude PP1 and the second amplitude PP2. Alternatively, the second critical range may range from a value smaller than the first amplitude PP1 by a constant value to a value greater than the second amplitude PP2 by a constant value. In addition, the processor 800 may set the second critical range to a preset value so as to calculate blood pressure information based on the measured first pulse wave signal PPG1.

Accordingly, if the abnormal section is not detected in the first pulse wave signal PPG1 (Y in S410), the processor 800 displays the first indicator (IN1 in FIG. 17) on the display panel DSP (S510). In addition, if the abnormal section is detected in the first pulse wave signal PPG1 (N in S410), the processor 800 displays the second indicator (IN2 in FIG. 19) on the display panel DSP (S520). This will be described later with reference to FIGS. 17 to 19.

In various embodiments, the processor 800 generates blood pressure information based on the pressure signal PSS and the first pulse wave signal PPG1 (S600), and displays the blood pressure information on the display panel DSP (S700). The processor 800 may generate a peak detection signal based on peak values of a third pulse wave signal PPG3. In addition, the processor 800 may calculate user's blood pressure information based on a peak value of the peak detection signal. This will be described later with reference to FIG. 23.

In various embodiments, the electronic device 1 may measure the pressure signal PSS and the first pulse wave signal PPG1, respectively, and calculate the blood pressure information based thereon. The electronic device 1 may display the guide indicator GI on the display panel DSP to measure a uniformly increasing pressure signal PSS. In addition, the electronic device 1 may calculate the abnormal measurement section of the pressure signal PSS or the first pulse wave signal PPG1, and accordingly display the first indicator IN1 or the second indicator IN2 on the display panel DSP. Accordingly, the electronic device 1 may accurately measure the user's blood pressure.

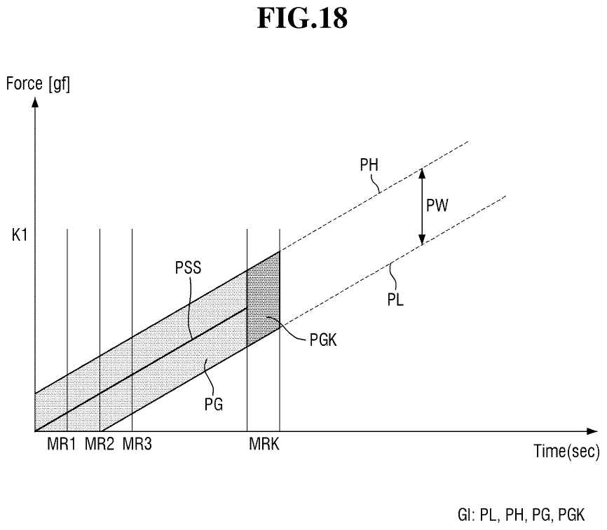

FIG. 17 is a schematic view illustrating a display area of the electronic device 1 according to an embodiment. FIG. 18 is an enlarged schematic view of a pressure guide PG. FIG. 19 is a schematic view illustrating a display area of the electronic device 1 according to an embodiment.

Referring to FIGS. 17 and 18, in an embodiment, the guide indicator GI may include an upper limit pressure PH, a lower limit pressure PL, and a pressure guide PG. The electronic device 1 according to an embodiment may control the shape of the pressure guide PG to gradually change over time so that the pressure applied to the pressure sensor SN_P (e.g., SN_P in FIG. 3) by a portion of the body of the user gradually increases. For example, in order to induce the pressure applied to the pressure sensor SN_P to gradually increase, the processor 800 may control a shape of the pressure guide PG to increase.

Referring to FIG. 18, the pressure guide PG may have a width of the first critical range PW1. For example, the pressure guide PG may have a value between the upper limit pressure PH and the lower limit pressure PL. That is, the pressure guide PG may be a pressure value in the first critical range PW1. In addition, the value of the pressure guide PG may gradually increase in the first to N-th measurement sections MR1 to MRN.

Accordingly, the user may apply pressure to the pressure sensor SN_P according to the pressure guide PG. For example, as illustrated in FIG. 18, when measuring pressure in a K-th measurement section MRK, a K-th pressure guide PG may be displayed on the display panel DSP. Accordingly, the user may apply pressure so that a pressure signal PSS of the K-th measurement section MRK is displayed in the K-th pressure guide PG.

In various embodiments, the processor 800 may control the generated pressure signal PSS to be displayed on the display panel DSP. The processor may check the pressure that the user applies to the pressure sensor SN_P in real time through the pressure signal PSS . When the pressure signal PSS is displayed in the pressure guide PG, the electronic device 1 may determine that desired pressure is applied. As described above, when the pressure guide PG gradually increases in length over time, and the pressure signal PSS is adjusted to be displayed within the width of the pressure guide PG, the pressure applied to the pressure sensor SN_P may be induced to increase or decrease to a certain level that can be a preset degree. The first critical range can have a constant pressure width that increases with time to a preset degree; and the processor is configured to display a guide indicator whose shape changes over time in response to the first critical range on the display panel.

Referring to FIGS. 17 and 19, the processor 800 may further display a first indicator IN1 or a second indicator IN2 on the display panel DSP.

As described above, when the abnormal measurement section is calculated in the pressure signal PSS or the first pulse wave signal PPG1, the electronic device 1 may not accurately calculate blood pressure information. Accordingly, when the abnormal measurement section is calculated in the pressure signal PSS or the first pulse wave signal PPG1, the processor 800 may display information on the abnormal measurement section on the display panel DSP. In addition, when the abnormal measurement section is not calculated in the pressure signal PSS or the first pulse wave signal PPG1, the processor 800 may display information on the same on the display panel DSP.

In various embodiments, the processor 800 may display different information on the display panel DSP according to the case in which the abnormal measurement section is calculated and the case in which the abnormal measurement section is not calculated. For example, when the abnormal measurement section is calculated in the pressure signal PSS or the first pulse wave signal PPG1, the processor 800 may display a first indicator IN1 on the display panel DSP. In various embodiments, the first indicator IN1 may be blue or green. In addition, when the abnormal measurement section is not calculated in the pressure signal PSS or the first pulse wave signal PPG1, the processor 800 may display a second indicator IN2 on the display panel DSP. In this case, the second indicator IN2 may be red.

In various embodiments, the first indicator IN1 and the second indicator IN2 may have the same shape. For example, the first indicator IN1 and the second indicator IN2 may extend along an edge of the display area. In addition, the first indicator IN1 and the second indicator IN2 may be displayed in a display area adjacent to the non-display area of the display panel DSP. Accordingly, the guide indicator GI may be displayed on a central portion of the display area, and the first indicator IN1 and the second indicator IN2 may be displayed on the edge of the display area.

In various embodiments, the electronic device 1 according to the embodiment displays the guide indicator GI and the pressure signal PSS in real time. Accordingly, the user may apply appropriate pressure to the pressure sensor SN_P according to the guide indicator GI and the pressure signal PSS displayed on the electronic device 1.

In various embodiments, the electronic device 1 displays the second indicator IN2 when the abnormal measurement section is calculated in the pressure signal PSS or the first pulse wave signal PPG1, and displays the first indicator IN1 when the abnormal measurement section is not calculated in the pressure signal PSS or the first pulse wave signal PPG1. That is, the user may determine in real time whether the electronic device 1 has measured the appropriate pressure signal PSS and first pulse wave signal PPG1.

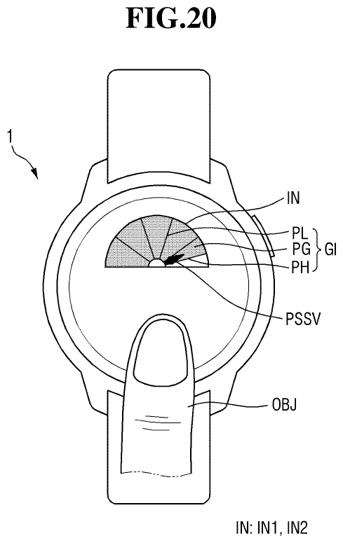

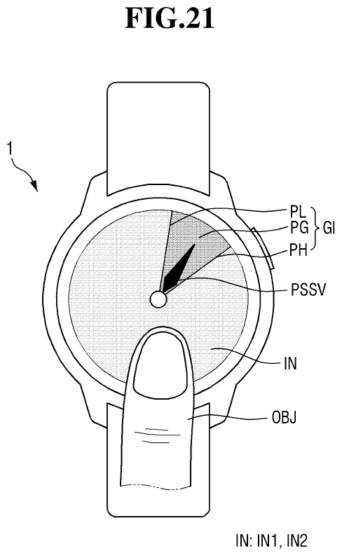

FIGS. 20 and 21 are schematic views illustrating display areas of the electronic device 1 according to an embodiment.

Referring to FIGS. 20 and 21, the pressure guide PG according to an embodiment may include a pressure guide PG displayed in a partial shape of a circle and a pressure signal value PSSV displayed in an arrow shape.

In various embodiments, the electronic device 1 may control the shape of the pressure guide PG to gradually change over time so that the pressure applied to the pressure sensor SN_P (e.g., SN_P in FIG. 3) by a portion of the body of the user gradually increases. For example, in order to induce the pressure applied to the pressure sensor SN_P to gradually increase, the processor 800 may control the shape of the pressure guide PG to increase.

In various embodiments, the pressure guide PG may be controlled so that an area thereof is changed according to the pressure applied to the pressure sensor SN_P. For example, the electronic device 1 may control the area of the pressure guide PG to increase in a clockwise direction as the pressure applied to the pressure sensor SN_P increases. The user may check the pressure that the user applies to the pressure sensor SN_P in real time through the pressure guide PG, wherein the pressure guide indicates the applied pressure. When the pressure signal value PSSV is displayed in the pressure guide PG, the electronic device 1 may determine that desired pressure is applied. As described above, when the area of the pressure guide PG gradually increases with time, and the pressure signal value PSSV equally follows the area change of the pressure guide PG, the pressure applied to the pressure sensor SN_P may be induced to increase or decrease to a certain level. In various embodiments, the pressure level currently applied to the pressure sensor SN_P may be converted into a numerical value and displayed in real time.

In the above, although the circular pressure guide PG has been described as an example of the pressure guide PG, the shape of the pressure guide PG may be variously changed into a shape that may induce a user to apply gradually increasing pressure to the display. For example, the pressure guide PG may have a partial shape of a circle or a partial shape of a donut, as in the example of FIG. 20. Alternatively, as in the example of FIG. 21, the pressure guide PG may be displayed in the entire area of the display area.

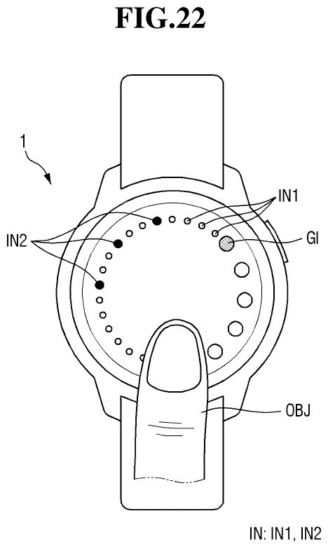

FIG. 22 is a schematic view illustrating a display area of the electronic device 1 according to an embodiment.

Referring to FIG. 22, the pressure guide PG according to an embodiment may include a pressure guide PG and a pressure signal value PSSV displayed in a shape in which a plurality of circles are arranged.

In various embodiments, the electronic device 1 may control the shape of the pressure guide PG to gradually change over time so that the pressure applied to the pressure sensor SN_P (e.g., SN_P in FIG. 3) by a portion of the body of the user gradually increases. For example, in order to induce the pressure applied to the pressure sensor SN_P to gradually increase, the processor 800 may control the shape of the pressure guide PG to increase.

In various embodiments, the pressure guide PG may be controlled so that an area and a position thereof are changed according to the pressure applied to the pressure sensor SN_P. For example, the pressure guide PG may have a shape in which a plurality of circles are arranged along the edge of the display area. In this case, each of the plurality of circles of the pressure guide PG may correspond to each of the measurement sections for measuring the pressure. Therefore, when the electronic device 1 senses the pressure, the pressure guide PG may be displayed by changing a shape or color of a circle corresponding to the measurement section among the plurality of circles.

In various embodiments, the first indicator IN1 and the second indicator IN2 may be displayed in the pressure guide PG. For example, when the abnormal measurement section exists in the pressure signal PSS or the first pulse wave signal PPG1, the second indicator IN2 may be displayed by changing a color of a circle corresponding to the abnormal measurement section. In this case, the second indicator IN2 may be red. For example, when the abnormal measurement section does not exist in the pressure signal PSS or the first pulse wave signal PPG1, the first indicator IN1 may be displayed by changing a color of a circle corresponding to the abnormal measurement section. In various embodiments, the first indicator IN1 may be blue or green.

In various embodiments, the user may also check the pressure that the user applies to the pressure sensor SN_P in real time through the pressure guide PG. When the shape of the pressure guide PG gradually moves with time, and the pressure signal value PSSV equally follows the shape change of the pressure guide PG, the pressure applied to the pressure sensor SN_P may be induced to increase or decrease to a certain level. According to various embodiments, the pressure level currently applied to the pressure sensor SN_P may be converted into a numerical value and displayed in real time.

In the above, although the circular pressure guide PG has been described as an example of the pressure guide PG, the shape of the pressure guide PG may be variously changed into a shape that may induce a user to apply gradually increasing pressure to the display.

FIG. 23 is a flowchart illustrating a method of calculating blood pressure according to an embodiment. FIG. 24 is a graph illustrating a waveform of a peak detection signal. A method of calculating blood pressure based on the third pulse wave signal PPG3 will be described with reference to FIGS. 23 and 24.

Referring to FIGS. 23 and 24, first, the processor 800 generates a third pulse wave signal PPG3 based on the pressure signal PSS and the first pulse wave signal PPG1 (ST1). In addition, the processor 800 generates a peak detection signal PPS (ST2).

In various embodiments, the processor 800 generates a third pulse wave signal PPG3 having the magnitude of the pulse wave signal according to the pressure based on the pressure signal PSS and the first pulse wave signal PPG1. In addition, the processor 800 may calculate an amplitude at each cycle T of the third pulse wave signal PPG3. In various embodiments, the processor 800 may generate the peak detection signal PPS having the magnitude of the third pulse wave signal PPG3 based on the amplitude of each cycle T of the third pulse wave signal PPG3. For example, the peak detection signal PPS can be a signal corresponding to the amplitude of each one cycle of the third pulse wave signal PPG3. That is, the peak detection signal PPS may be defined as a signal corresponding to a peak value of each one cycle of the third pulse wave signal PPG3. For example, the third pulse wave signal PPG3 may have at least one or more amplitudes. The processor 800 may calculate the peak detection signal PPS including points corresponding to the amplitude of each cycle T of the third pulse wave signal PPG3. That is, the generated peak detection signal PPS may be a signal having the amplitude according to the pressure.

In various embodiments, the processor 800 determines whether a pressure value corresponding to a peak value PK of the peak detection signal PPS may be calculated (ST3). When a peak of the peak detection signal PPS exists, the processor 800 may calculate the pressure value corresponding to the peak value PK of the peak detection signal PPS.

In various embodiments, the processor 800 calculates a systolic blood pressure SBP, a diastolic blood pressure DBP, and the like based on the peak value PK of the peak detection signal PPS (ST4), and calculates blood pressure information (ST5).

In various embodiments, the processor 800 may calculate the diastolic blood pressure DBP lower than the pressure value, the systolic blood pressure SBP higher than the pressure value, and a mean blood pressure according to the pressure value. For example, the processor 800 may calculate pressure values corresponding to values corresponding to 60% to 80% of the peak value PK. The processor 800 may calculate a pressure value smaller than a pressure value corresponding to the peak value PK among the pressure values as a first pressure value PR1. In addition, the processor 800 may calculate the first pressure value PR1 as the diastolic blood pressure DBP. The processor 800 may calculate a pressure value greater than the pressure value corresponding to the peak value PK among the pressure values as a second pressure value PR2. In addition, the processor 800 may calculate the second pressure value PR2 as the systolic blood pressure SBP.

In the embodiment, since the third pulse wave signal PPG3 vibrates according to a heartbeat cycle, the third pulse wave signal PPG3 may reflect a change in blood pressure according to the heartbeat. The electronic device may accurately calculate blood pressure information based on the third pulse wave signal PPG3.

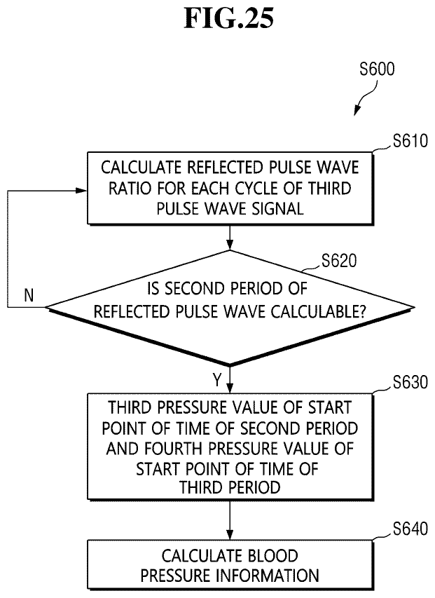

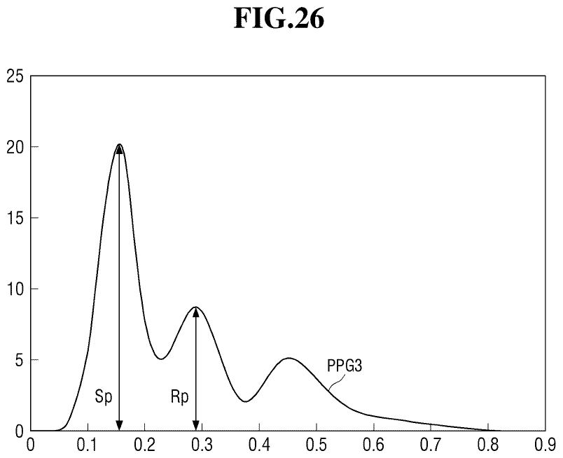

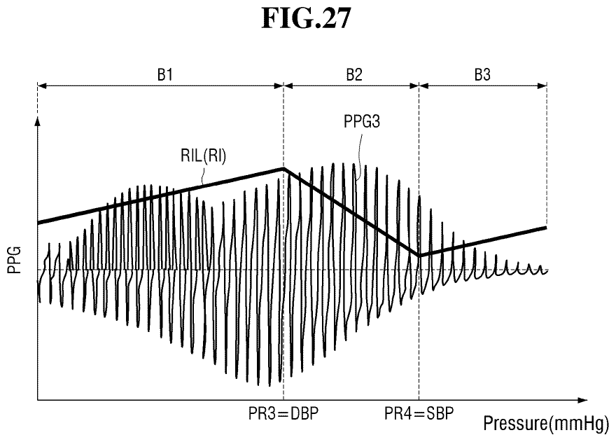

FIG. 25 is a flowchart illustrating a method of calculating blood pressure according to another embodiment. FIG. 26 is a graph illustrating a waveform of one cycle of a pulse wave signal according to another embodiment. FIG. 27 is a graph illustrating a pulse wave signal and a reflected pulse wave ratio according to another embodiment. Hereinafter, a method in which a display device calculates blood pressure based on a reflected pulse wave ratio RI will be described with reference to FIGS. 25 to 27.

Referring to FIG. 25, first, a reflected pulse wave ratio RI is calculated for each cycle of the third pulse wave signal PPG3 (S610).

Referring further to FIG. 26, in order to calculate the reflected pulse wave ratio RI, the processor 800 divides a wave cycle of the third pulse wave signal PPG3 according to a period in which a wave according to a heartbeat and a reflected wave of a blood vessel are sequentially generated. For example, one cycle of the third pulse wave signal PPG3 may include a plurality of waveforms having different amplitudes. Accordingly, a peak value of a waveform having the largest amplitude among the plurality of waveforms may be defined as a pulse wave contraction value, a peak value of a waveform having the second largest amplitude among the plurality of waveforms may be defined as a reflected pulse wave value, and when the pulse wave contraction value is defined as Sp, the reflected pulse wave value is defined as Rp, and the reflected pulse wave ratio is defined as RI, the reflected pulse wave ratio RI may be calculated by Equation 1 below.

$$R I = \frac{Rp}{Sp} \qquad [ Equation 1 ]$$

Here, the pulse wave contraction value Sp may have the same value as the main amplitude of each of the first to N-th measurement sections. The peak value Rp of the waveform having the second largest amplitude may be substantially the same as sub-amplitude of each of the first to N-th measurement sections.

In various embodiments, the processor 800 may calculate the peak value of the waveform having the largest amplitude among the plurality of waveforms of one cycle of the third pulse wave signal PPG3. In addition, the processor 800 may calculate the peak value of the waveform having the second largest amplitude among the plurality of waveforms of one cycle of the third pulse wave signal PPG3. In addition, the processor 800 may calculate the reflected pulse wave ratio RI based on the pulse wave contraction value Sp and the reflected pulse wave value Rp.

In various embodiments, the processor 800 determines whether a second period B2 of the reflected pulse wave ratio RI may be calculated (S420). The processor 800 sequentially stores a detection result of the pulse wave contraction value to the reflected pulse wave ratio RI, and analyzes the stored reflected pulse wave ratio RI. The processor 800 may analyze a change in the magnitude of reflected pulse wave ratio data RIL(RI) by continuously dataizing a change in the magnitude of the reflected pulse wave ratio RI.

The reflected pulse wave ratio RI includes a first period B1 in which the reflected pulse wave ratio RI fluctuates within a first range, a second period B2 in which the reflected pulse wave ratio RI fluctuates within a second range, and a third period B3 in which the reflected pulse wave ratio RI fluctuates within a third range. For example, the processor 800 may analyze a first period B1 in which the reflected pulse wave ratio RI is less variably changed within a preset range in a saturated state, a second period B2 in which the reflected pulse wave ratio RI is rapidly lowered or higher than the preset range within a preset period, and a third period B3 in which the reflected pulse wave ratio RI is rapidly lowered or higher and then is less variably changed within the preset range in the saturated state by analyzing a reflected pulse wave ratio signal RIL.

Here, a width of the first range and a width of the third range may be smaller than a width of the second range. In addition, a slope of the second period B2 of the reflected pulse wave ratio RI may be greater than a slope of the first period B1 of the reflected pulse wave ratio RI and a slope of the third period B3 of the reflected pulse wave ratio RI.

In various embodiments, the processor 800 calculates a systolic blood pressure SBP, a diastolic blood pressure DBP, and the like based on the reflected pulse wave ratio RI (S630), and calculates blood pressure information (S640).

In various embodiments, the processor 800 may analyze the reflected pulse wave ratio RI to detect a start point of time at which the second period B2 starts. In addition, the processor 800 may calculate a third pressure value PR3 corresponding to the third pulse wave signal PPG3 at the start point of time of the second period B2. The processor 800 may calculate the third pressure value PR3 as the diastolic blood pressure DBP. In addition, the processor 800 may analyze the reflected pulse wave ratio RI to detect a start point of time at which the third period B3 starts after the second period B2. In addition, the processor 800 may calculate a fourth pressure value PR4 corresponding to the third pulse wave signal PPG3 at a point of time at which the third period B3 starts. The processor 800 may calculate the fourth pressure value PR4 as the systolic blood pressure SBP.

In the embodiment, since the third pulse wave signal PPG3 vibrates according to a heartbeat cycle, the third pulse wave signal PPG3 may reflect a change in blood pressure according to the heartbeat. The electronic device may accurately calculate blood pressure information based on the third pulse wave signal PPG3.

In concluding the detailed description, those skilled in the art will appreciate that many variations and modifications can be made to the preferred embodiments without substantially departing from the principles of the invention. Therefore, the disclosed preferred embodiments of the invention are used in a generic and descriptive sense only and not for purposes of limitation.

What is claimed is:

1. An electronic device comprising:
a display panel;
a light sensor disposed on the display panel;
a pressure sensor configured to sense an applied pressure; and
a processor configured to receive a pressure signal from the pressure sensor and a first pulse wave signal from the light sensor,
wherein the processor is configured to:
analyze the pressure signal;
display a first indicator on the display panel in response to a first abnormal section being calculated from the pressure signal;
display a second indicator different from the first indicator on the display panel in response to the first abnormal section not being calculated; and
calculate blood pressure information based on the first pulse wave signal and the pressure signal.

2. The electronic device of claim 1,
wherein the processor is configured to analyze the first pulse wave signal to display the first indicator on the display panel in response to a second abnormal section being calculated based on the first pulse wave signal.

3. The electronic device of claim 2,
wherein the processor is configured to calculate at least one measurement section as the first abnormal section in response to a magnitude of the pressure signal in the at least one measurement section among the measurement sections of the pressure signal does not exist within a first critical range.

4. The electronic device of claim 3,
wherein the first critical range has a constant pressure width and increases with time to a preset degree; and
the processor is configured to display a guide indicator whose shape changes over time in response to the first critical range on the display panel.

5. The electronic device of claim 4,
wherein the processor is configured to display the magnitude of the pressure signal on the guide indicator so that at least one of a shape, a color and a location changes over the time.

6. The electronic device of claim 2,
wherein the processor is configured to calculate at least one measurement section as the second abnormal section when a magnitude of the first pulse wave signal in the at least one measurement section among measurement sections of the first pulse wave signal is not within a second critical range.

7. The electronic device of claim 1,
wherein the display panel includes a display area in which pixels and the light sensor are disposed, and a non-display area disposed at one side of the display area; and
wherein at least one of the first indicator and the second indicator has a shape extending along an edge of the display area.

8. The electronic device of claim 7,
wherein the first indicator and the second indicator have the same shape.

9. The electronic device of claim 1,
wherein the processor is configured to further calculate a third pulse wave signal including a pulse wave signal value according to pressure based on the first pulse wave signal and the pressure signal.

10. The electronic device of claim 9,
wherein the processor is configured to:
generate a peak detection signal based on an amplitude corresponding to a peak of each cycle of the third pulse wave signal;
calculate a peak value of the peak detection signal and a pressure value corresponding to the peak value of the peak detection signal and calculate a diastolic blood pressure lower than the pressure value, a systolic blood pressure higher than the pressure value, and a mean blood pressure according to the pressure value; and
display the diastolic blood pressure and the systolic blood pressure on the display panel.

11. The electronic device of claim 10,
wherein the processor is configured to calculate the mean blood pressure as a pressure value corresponding to the peak value.

12. The electronic device of claim 10,
wherein the processor is configured to:
calculate a first pressure value smaller than the pressure value corresponding to 60% to 80% of the peak value and a second pressure value greater than the pressure value in the peak detection signal; and
calculate the first pressure value as the diastolic blood pressure and calculate the second pressure value as the systolic blood pressure.

13. The electronic device of claim 9,
wherein one cycle of the third pulse wave signal includes a plurality of waveforms having different amplitudes; and
a peak value of a first waveform among the plurality of waveforms is defined as a pulse wave contraction value, a peak value of a second waveform among the plurality of waveforms is defined as a reflected pulse wave value, and when the pulse wave contraction value is defined as Sp, the reflected pulse wave value is defined as Rp, and a reflected pulse wave ratio is defined as Ri, the processor is configured to calculate the reflected pulse wave ratio by
$$R I = \frac{Rp}{Sp} .$$

14. The electronic device of claim 13,
wherein the reflected pulse wave ratio includes a first period in which the reflected pulse wave ratio fluctuates within a first range, a second period in which the reflected pulse wave ratio fluctuates within a second range, and a third period in which the reflected pulse wave ratio fluctuates within a third range; and
a width of the first range and a width of the third range are smaller than a width of the second range.

15. The electronic device of claim 14,
wherein the processor is configured to:
analyze the reflected pulse wave ratio to detect a start point of time at which the second period starts;
calculate a third pressure value corresponding to the first pulse wave signal at the start point of time of the second period;
set the third pressure value as a diastolic blood pressure;
calculate a fourth pressure value corresponding to the first pulse wave signal at a point of time at the third period starts after the second period; and
calculate the fourth pressure value as a systolic blood pressure.

16. A method of operating an electronic device, including a display panel, a light sensor, a pressure sensor, and a processor comprising:
calculating a first abnormal section based on a pressure signal;
displaying a first indicator on the display panel in response to the first abnormal section being calculated from the pressure signal;
displaying the first indicator on the display panel in response to the first abnormal section being calculated from the pressure signal;
displaying a second indicator different from the first indicator on the display panel in response to the first abnormal section not being calculated; and
calculating blood pressure information based on the first pulse wave signal and the pressure signal.

17. The method of claim 16,
wherein in the calculating of the first abnormal section by analyzing the pressure signal, when a magnitude of the pressure signal in at least one measurement section among the measurement sections of the pressure signal does not exist within a first critical range, the at least one measurement section is calculated as the first abnormal section.

18. The method of claim 17, further comprising,
displaying a guide indicator so that at least one of a shape, a color and a location changes over time in response to the first critical range on the display panel,
wherein the first critical range has a constant pressure width and increases with time to a preset degree.

19. The method of claim 16, further comprising
calculating a second abnormal section by analyzing a first pulse wave signal;
wherein in the calculating of the second abnormal section by analyzing the first pulse wave signal,
when a magnitude of the first pulse wave signal in at least one measurement section among measurement sections of the first pulse wave signal does not exist within a second critical range, the at least one measurement section is calculated as the second abnormal section.

20. The method of claim 16,
wherein the display panel includes a display area in which pixels and the light sensor are disposed and a non-display area disposed at one side of the display area, and
at least one of the first indicator and the second indicator has a shape extending along an edge of the display area.

21. An electronic device comprising:
a display panel;
a light sensor disposed on the display panel;
a pressure sensor configured to sense an applied pressure; and
a processor configured to receive a pressure signal from the pressure sensor and a first pulse wave signal from the light sensor,
wherein the processor is configured to:
calculate blood pressure information based on the first pulse wave signal and the pressure signal, and determine that a magnitude of the pressure signal is not within a first critical range.

22. The electronic device of claim 21, further comprising:
a driving unit configured to activate the pressure sensor.

23. The electronic device of claim 22, wherein the driving unit is further configured to transmit a driving signal to the light sensor PS to activate the light sensor PS.

24. The electronic device of claim 22, further comprising:
a touch sensor configured to detect a touch event; and
an indicator generation unit configured to generate a guide indicator on the display panel DSP, wherein the guide indicator includes a pressure guide configured to indicate the applied pressure.
